(19) United States
(12) Patent Application Publication
(10) Pub. No.: US 2006/0264935 A1
(43) Pub. Date: Nov. 23, 2006

(54) Orthopedic stabilization device

(76) Inventor: Patrick M. White, West Chester, PA (US)

Correspondence Address: Patrick M. White, 511 Price Street, West Chester, PA, 19382, US

(21) Appl. No.: 11/244,184

(22) Filed: Oct. 5, 2005

Related U.S. Application Data
(60) Provisional application No. 60/677,699, filed on May 4, 2005.

(51) Int. Cl. A61F 2/30

(52) U.S. Cl. 606/61

(57) ABSTRACT

An orthopedic device is described for stabilizing the spinal column between first and second vertebral bodies. The device has first and second anchors adapted for fixation to the first and second vertebral bodies, respectively. The device further includes an elongated bridge with first and second ends operatively connected to the first and second anchors, respectively. The bridge contains an implantable ligament made preferably of a nickel titanium alloy selected to have inelastic properties at body temperature, wherein the bridge is capable of continuous plastic deformation to allow relative constrained motion between the vertebral bodies. In a preferred embodiment the device further includes a dampening member surrounding the ligament, which may be a wire, tube or band. In another preferred embodiment the device includes rigid rod members each correspondingly retained with either end of the ligament, and independently attached to the vertebral bodies with anchors. The rigid rod members are correspondingly connected to either end of the ligament. In yet another preferred embodiment, the device includes plate segments correspondingly retained with either end of the ligament, and independently attached to the vertebral bodies with a plurality of anchors. Other preferred devices and kits containing such devices are also described.

### RELATED APPLICATION

This application claims priority from U.S. Provisional Application No. 60/677,699, filed May 4, 2005 by the present inventor.

### BACKGROUND OF THE INVENTION

1. Technical Field of the Invention

The present invention relates to orthopedic stabilization devices used to limit the relative motion of at least two vertebral bodies for the relief of pain. These devices can be used to aid osteo-synthesis in combination with fusion devices, supplement other motion restoring devices such as disk implants or used solely to restrict the motion of vertebral bodies without other devices.

2. Description of the Related Art

In the field of spine surgery there have been many attempts to relieve pain associated with spinal injury or illness. Traditionally surgeons have fused the vertebral bodies with a pedicle screw and rod construct or a fusion cage. In attempting to fuse the patient there is a long and painful recovery process. Most rod and screw constructs and fusion cage constructs are very rigid, not allowing transfer of stress into the fusion site that would otherwise aid in a quicker recovery. These approaches defy Wolfe's law stating: bone that is not stressed will degrade. As a corollary, where stress is allowed to transfer through the fusion site while the vertebral bodies are held in a limited range of motion, then fusion can occur much quicker aiding in patient recovery time.

Many are working to develop devices that allow relative motion, yet these have fallen short in preventing shear forces between the vertebral bodies being stabilized. Another shortcoming is that relative motion has been forcibly channeled through a rather specific location or hinge point in the mechanical construct. The following discussion more particularly summarizes these efforts.

U.S. Pat. No. 5,092,866 (U.S. Re. 36,221) discloses a pedicle screw system that is banded together with flexible ligaments. While the ligaments allow for relative motion, they do not appear to resist compression or shear loads, instead relying upon tension alone.

European Patent No. EP 06691091 A1/B1 and the “DYNESYS” product brochure disclose a polycarbonate/urethane supporting element, compressed between two adjacent pedicle screws and passing over an elastic strap that acts as a flexible internal ligament. The flexible internal ligament is in the form of a nylon cord, which is pre-tensioned and fastened to the screw heads. This design provides flexural degrees of freedom, allows relative motion between the vertebral bodies but does little to inhibit or prevent shearing between the vertebral bodies. While flexibility is desirable, the “DYNASES” ligament would appear to lack rigidity and rely on proper tensioning inter-operatively to gain its balance.

U.S. Pat. No. 6,267,764 discloses a pedicle screw and rod system wherein the rod is flexible in translation. A dampening ball is not separate from the rods and has cutouts to allow bending, with no ligament passing through the centers of the rods. While flexibility in translation can be helpful, the spine loads in several planes at the same time and the translation spoken of in this patent would appear to inadequately redistribute stresses through the fusion site. As a result motion is forcibly limited to one location, i.e., motion is constrained through a hinge point, which undesirably stresses the assembly construct.

U.S. Pat. No. 6,241,730 discloses a construction that lacks a ligament element, particularly a ligament extending through the center of rod members. There is a compressible dampening element. The '730 design attempts to accomplish a multidirectional redistribution of force for aiding in quicker fusion rates, however its constructs were not designed for use in conjunction with a disk implant. The '730 approach overly limits motion of the vertebral bodies to one location, i.e., forces motion unnaturally through a hinge point.

U.S. Pat. Nos. 6,293,949 and 6,761,719 disclose embodiments seeking to elastically constrain range of motion using a continuous super-elastic nitinol rod and pedicle screw system. Due to the super-elastic state of the rod, motion is always pushed-back to a neutral, pre-set position. This constrains force through the rod in a manner causing early fatigue failure. In order to provide the correct elasticity of the rod, its diameter must be so small that it cannot withstand the continuous loads. Further, the rod cannot be bent at the time of surgery to a preformed shape holding the vertebral bodies in a desired relative position while also limiting their relative motion.

Accordingly there exists a need for assemblies and devices that effectively resist torsion as well as shear forces while providing flexible spine stabilization. More specifically, it would be desirable to provide kits with such assemblies and devices, which work with existing pedicle screw arrangements.

There is another need for flexible assemblies and devices having rigid portions deformable to fit a patient's anatomical contours while maintaining flexibility of the orthopedic construct.

There is yet another need for assemblies and devices to stabilize vertebrae while providing multi-directional flexibility, without imparting elastic stresses to the bone.

There is a further need to provide spine stabilization assemblies and devices manufactured from a shape memory material such as an alloy or other flexible polymer, which can withstand repeated loading of the spine without fatiguing yet still maintain its flexibility.

### SUMMARY OF THE INVENTION AND ADVANTAGES

According to one embodiment of the present invention, there is provided an orthopedic device for stabilizing the spinal column between anchorage locations on respective first and second vertebral bodies. The device includes an elongated bridge having first and second ends operatively connected at the respective anchorage locations. The bridge contains an implantable ligament selected to be inelastic at body temperature. The ligament is further capable of continuous plastic deformation to allow relative constrained motion while resisting forces exerted upon the vertebral bodies. In a preferred embodiment, the bridge contains an implantable nickel titanium alloy. In another preferred embodiment the device further includes a dampening member surrounding at least a portion of the ligament. In yet another preferred embodiment, the ligament is in the form of a wire, tube, or band. In still another preferred embodiment the device includes rigid rod members each correspondingly retained with either end of the ligament, and independently attached to the vertebral bodies with anchors. The rigid rod members are correspondingly connected to either end of the ligament. In still yet another preferred embodiment, the device includes a plate segment retained with an end of the ligament and independently attached to a vertebral body with the plurality of anchors; more preferably, a plurality of plate segments are correspondingly connected to either end of the ligament.

In another embodiment of the present invention, an orthopedic device for stabilizing the spinal column includes an elongated implantable ligament with two ends, the ligament partially formed of an implantable nickel titanium alloy capable of continuous plastic in-elastic deformation at body temperature. Either end of the ligament is attached to a vertebral body with a screw at an anchor location. A compression-dampening member surrounds the ligament and is sandwiched between the screws. Plastic deformation in the ligament allows relative constrained motion between the vertebral bodies.

In yet another embodiment of the present invention, an orthopedic device for stabilizing the spinal column is disclosed. The device includes an implantable elongated ligament with two enlarged end portions. The ligament is partially formed of a nickel titanium alloy capable of continuous plastic in-elastic deformation at body temperature. Two rigid rod members each contain a bore sized for the ligament, the rigid rod members being retained with either end of the ligament and engageable with two vertebral bodies by a plurality of anchors. A compression-dampening member surrounds the ligament and is sandwiched between the rods. Two tension-dampening members are captured within the rigid rod bores, surround the ligament and abut the enlarged end portions respectively. Plastic deformation in the ligament allows relative constrained motion between the vertebral bodies.

In still another embodiment of the present invention, a surgical kit is disclosed. The kit includes at least one bone anchor and a flexible spine stabilization device. The device includes a ligament partially formed of an implantable nickel titanium alloy capable of continuous plastic in-elastic deformation at body temperature. In a preferred embodiment, the surgical kit includes at least one rigid fusion rod. The anchor, ligament and rigid fusion rod mentioned above are provided in various sizes to accommodate a given patient's anatomy.

An advantage of the present invention is a device that limits the range of relative motion between two vertebral bodies and works with existing pedicle screw assemblies.

Another advantage of the invention is to provide a kit to the surgeon that has a variety of pedicle screws, rigid fusion rods and elongated implantable inelastic ligaments. Further it is desirable that the ligaments provide a variety of stiffness and flexibility options so the surgeon can select the appropriate stiffness and range of motion to achieve the desired surgical result whether it is for aiding fusion or restoring normal range of motion to a patient.

Other objects and advantages will become apparent to a reader skilled in the art, with reference to the following Figures and accompanying Detailed Description wherein textual reference characters correspond to those denoted on the Drawings.

### BRIEF DESCRIPTION OF THE DRAWINGS

In the accompanying drawings:

FIG. 1 is an exploded perspective view of a flexible inelastic spine stabilization device, according to the present invention;

FIG. 1A is a perspective view of a representative plate segment for securing the device of FIG. 1;

FIG. 2 is a perspective view of the device of FIG. 1, shown in its assembled state;

FIG. 3 is an elevational view of the device of FIGS. 1 and 2, further including pedicle screws for attaching the device to adjacent vertebral bodies as schematically shown;

FIG. 4 is an elevational view of the device of FIG. 3, upon application of load;

FIG. 5 is a kit of spinal implant components including a pedicle screw, a rigid fusion rod, and a ligament of the present invention, selected from among various ranges of flexibility;

FIG. 6 is a top view of a device employing an elongated implantable ligament attached to vertebral bodies (schematically shown) with pedicle screws that directly secure the ligament between the screws;

FIG. 7 is an elevational view of the device of FIG. 6;

FIG. 8 is an exploded perspective view of another device of the present invention, employing a ligament surrounded by compression and tension-damping members;

FIG. 9 is an elevational view of the assembled structures shown in FIG. 8 prior to application of a load; and

FIG. 10 is a sectional view taken longitudinally along Lines 10-10 of FIG. 9.

### DETAILED DESCRIPTION OF THE INVENTION

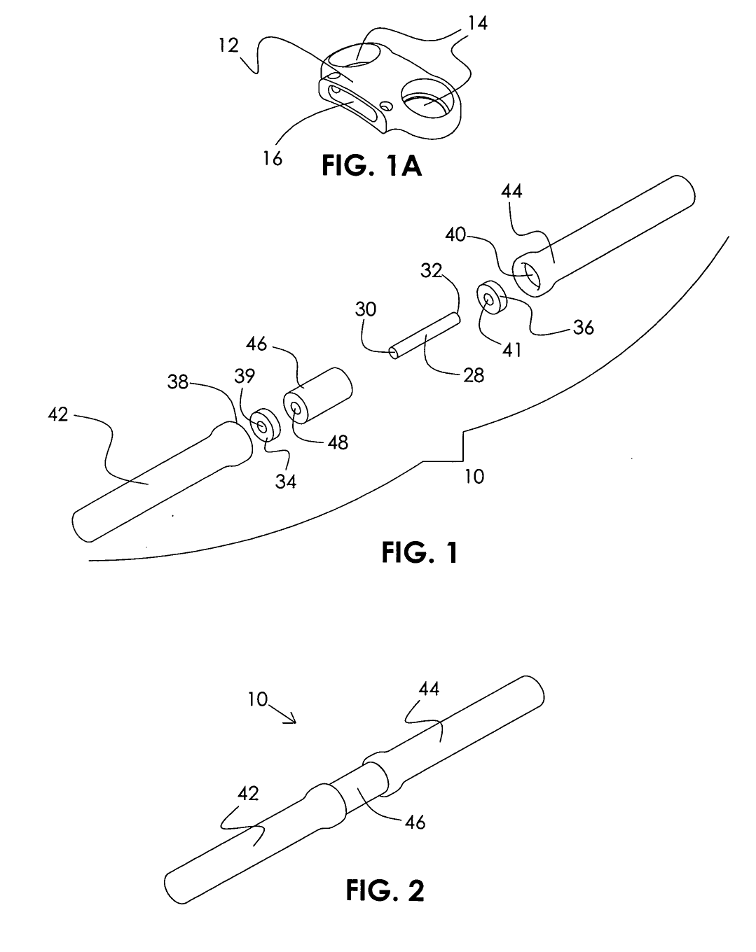

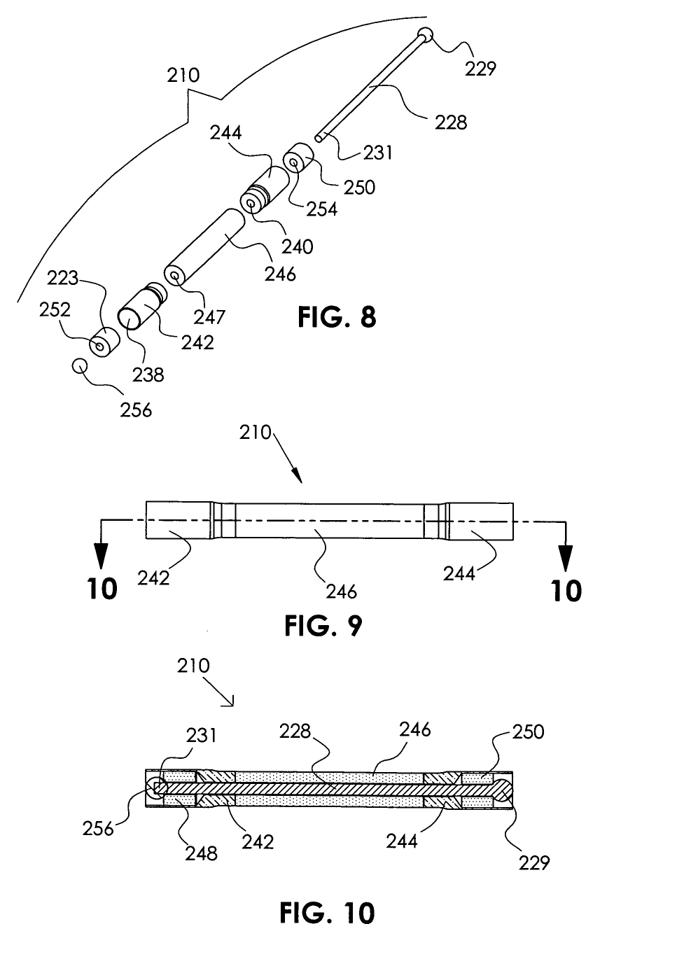

With reference generally to FIGS. 1-10, the Applicant's invention provides flexible spinal stabilization allowing controlled relative vertebral motion for the relief of pain, while preventing intervertebral shear forces. Moreover, the invention evenly distributes mechanical stresses throughout its structure rather than constraining motion within a limited portion of its structure, by virtue of its distinctive design.

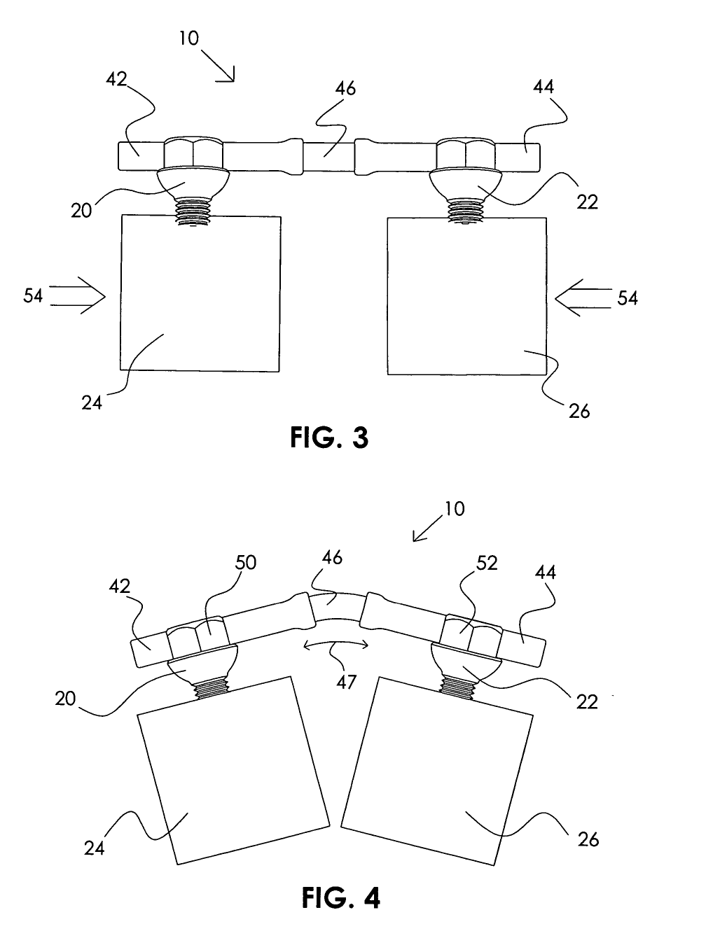

Referring to FIGS. 1 and 2-4, an elongated bridge member is generally shown as an assembly at 10. Assembly 10 includes a ligament 28 shown in the form of a wire. It will be understood that the ligament 28 may also take the form of a tube, solid rod or a band, having different cross sectional shapes and sizes. The ligament 28 is made of an implantable material selected to be inelastic at body temperature and allows relative constrained motion while resisting bodily shear forces. The ligament 28 has opposed first 30 and second 32 ends received within washer type connectors 34, 36 that engage counter-bores 38, 40 formed within rigid rod members 42, 44, respectively. Those in the art will appreciate that the rigid rod members 42, 44 could have differing sizes and/or lengths. Washer-shaped connectors 34, 36 are preferably made of a shape-memory alloy in its super-elastic state at body temperature. Alternatively other means for attaching the in-elastic ligament 28 to rigid rod potions 42, 44 may include welding, threading, gluing or crimping instead of using connectors 34, 36. Thus, assembly 10 operatively functions as a bridge between a first anchor 20 and a second anchor 22, respectively. Ligament 28 is preferably made of an implantable shape memory alloy, more preferably a nickel titanium alloy, which is selected to be inelastic at body temperature. That is, ligament 28 is not in a super-elastic state. Preferably, assembly 10 may include a dampening member 46 that has an inner diameter 48 surrounding ligament 28. Referring to FIGS. 3-4, first and second screw anchors 20, 22 are adapted for respectively affixing assembly 10 to first and second vertebral bodies 24, 26.

Referring to FIG. 1A, a representative plate segment 12 has openings 14 that receive anchoring screws for attachment to a vertebral body not shown. Each plate segment 12 has a passageway 16 configured to receive one end of a ligament not shown. It will be understood that the ligament used in conjunction with the plate 12 could have a variety of forms as elucidated in the above discussion of FIGS. 1 and 2-4. Passageway 16 could have a rectangular cross section as shown, or could have a variety of forms for receiving the ligament. Preferably a plurality of plates 12 can be employed with the ligament, across a corresponding plurality of vertebral bodies to form a bridge similar to assembly 10 in FIGS. 1 and 2-4.

Referring to FIGS. 3-4, plastic deformation in ligament 28 is in response to external stimulus indicated by arrows 54, 54, which for the sake of illustration is shown as direct uniform axial compression. However, as will be appreciated, the external stimulus often consists of combined bending and twisting motions of a patient's body.

With continuing reference to FIGS. 1-4, the present assembly 10 resists shear forces exerted between vertebral bodies 24, 26 during the bending and twisting motions of a patient without creating elastic forces that otherwise would exert unnatural stresses forcing the vertebral bodies back into some prior position. The present invention instead allows the body's own motion to return it to the natural position without undue elastic impetus. This natural return to body position is therefore distinct from prior approaches that rely upon super-elastic members such as those discussed above; moreover, the present invention is distinct from prior approaches that do not resist both shear and direct torsional movements while yet bending themselves. The present assembly 10 does not exert resultant forces that are opposite to the motion input 54, 54 and yet the assembly is repetitively plastically deformable due to the material and design employed herein.

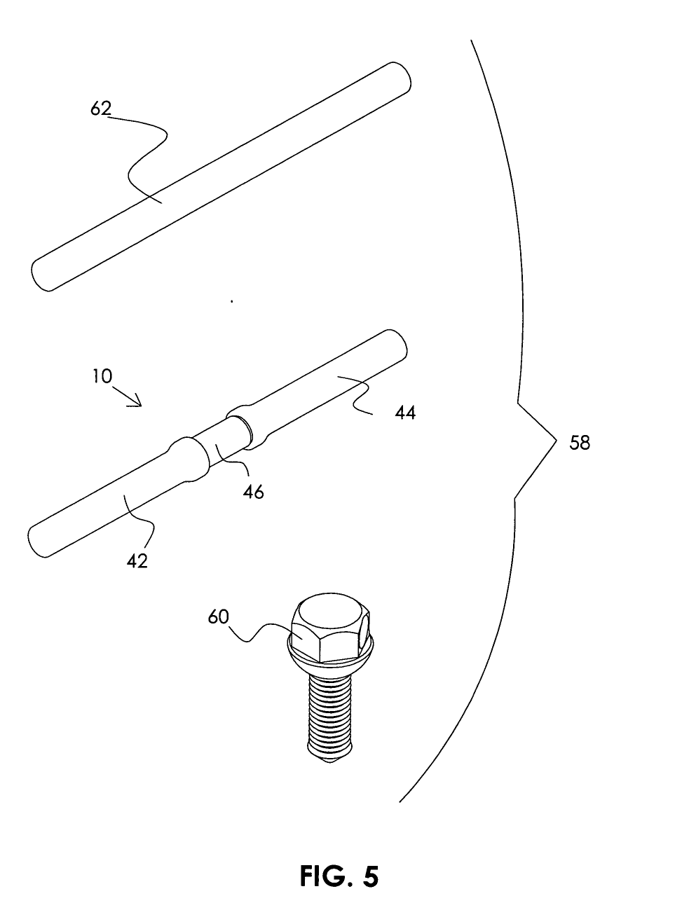

FIG. 5 depicts still another embodiment of the present invention, that is, a surgical kit generally shown at 58. Kit 58 includes an array of bone anchors 60 and an elongated bridge assembly 10 preferably of the type shown in FIGS. 1-4 although it will be appreciated that an array of assemblies having various sizes and stiffness can be provided. The assembly 10 is capable of continuous plastic in-elastic deformation at body temperature. In a preferred embodiment, the surgical kit 58 includes an array of semi-rigid fusion rods such as the representative rod shown at 62. In another preferred embodiment, an alternative surgical kit not shown may include an array of plates similar to those described in conjunction with FIG. 1A. The arrays mentioned above are provided in various sizes to accommodate a given patient's anatomy.

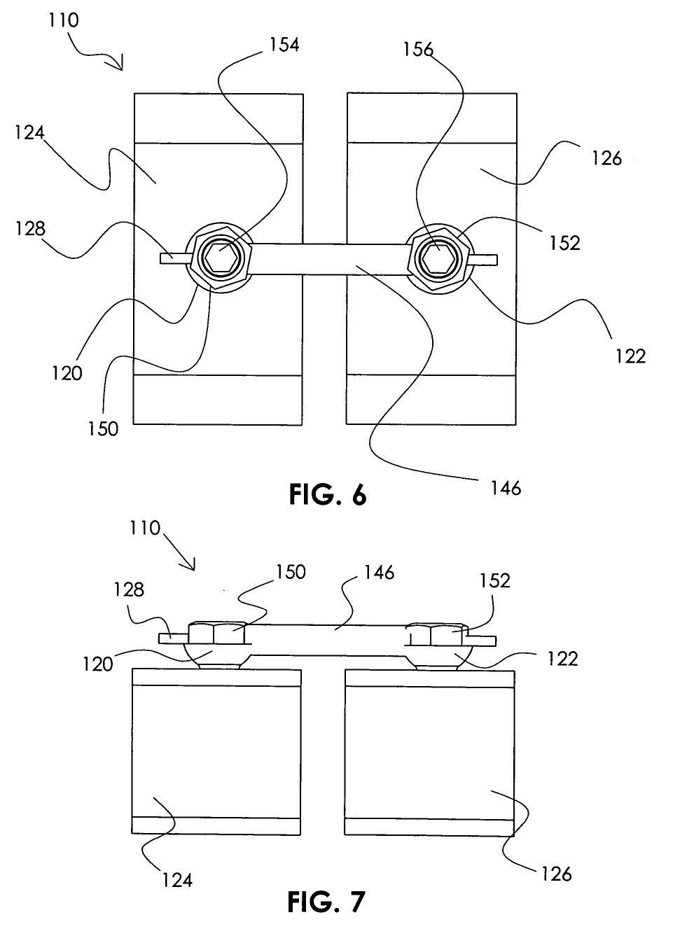

Referring to FIGS. 6-7, an orthopedic device 110 for stabilizing the spinal column includes an elongated bridge member that takes the form of ligament 128 instead of the assembly 10 as previously discussed in conjunction with FIGS. 1 and 2-4. It will be understood that the ligament 128 may also take the form of a tube, solid rod or a band, having different cross sectional shapes and sizes. The ligament 128 is at least partially made of an implantable material that is preferably a nickel titanium alloy capable of continuous plastic in-elastic deformation at body temperature in similar fashion as ligament 28 (FIG. 1). Device 110 has no distinct rigid rod members as depicted, for example, at 42 and 44 in FIGS. 1 and 2-4. Nor are there any plate segments as at 12 in FIG. 1A to operatively anchor ligament 28. Instead, ligament 128 extends between and directly interconnects screws 120, 122, which affix it to vertebral bodies 124 and 126. An optional compression-dampening member 146 is shown surrounding ligament 126 and is sandwiched between the screw heads 150, 152 in FIGS. 6-7. Plastic deformation in the ligament 128 allows relative motion while preventing shear stresses between vertebral bodies 124, 126. FIGS. 6-7 show a tubular dampening member 146 preferably made of an in implantable elastomer such as silicone or polycarbonate urethane, through which elongated ligament 128 passes.

Referring to FIGS. 8-10 there is yet another embodiment of the present invention. An elongated bridge member is shown in the form of an assembly 210, for stabilizing the spinal column. Assembly 210 includes an elongated ligament 228 with an enlarged fixed end portion 229 and a free end 231. Ligament 228 is at least partially formed of an implantable inelastic material, preferably nickel titanium alloy capable of continuous plastic in-elastic deformation at body temperature, i.e., not in a super-elastic state. Bore 240 of rigid rod member 244 is sized for passage of ligament 228, the rod members 242, 244 are retained with ends 229, 231 of the ligament for attachment at respective anchorage locations to vertebral bodies (not shown). Those in the art will appreciate that the rigid rod members 242, 244 could have differing sizes and/or lengths. A compression-dampening member 246 has a bore 247 that surrounds ligament 228 and is sandwiched between proximally chamfered rigid rod members 242, 244. Tension-dampening members 248, 250 have respective bores 252, 254 sized to allow passage of ligament 228 housed within the bores of the rigid rods 242, 244. Tension-dampening members 248, 250 surround ligament 228 and are respectively captured by rigid rods 242, 244 along with enlarged ends 229, 256 as shown in FIG. 10. Plastic deformation in ligament 228 allows relative constrained motion between, while resisting shear forces exerted upon the vertebral bodies. Motion is transmitted along the entire length of ligament 228 from its enlarged fixed ends 229, 256. Dampening members 246, 248, 250 are preferably made of an implantable elastomer such as silicone or polycarbonate urethane

Inelastic ligament 28, 128, 228 is preferably manufactured from a nickel titanium alloy preferably having a diameter in the range of 3-6 mm. Other cross sectional shapes and sizes of ligament 28, 128, 228 may be made available for different surgical applications. Nickel Titanium can be alloyed to have varying properties, some alloys exhibiting super-elastic behavior at body temperature while other alloys are continuously in-elastic at body temperature. These inelastic alloys are commonly referred to as shape memory alloys by those skilled in the art. Shape memory alloys may further have transition temperatures either above or below body temperature; however, the applicable transition temperature for the present invention is selected to be higher than body temperature. The present inventor has determined that within the operative size range, in-elastic ligaments 28, 128, 228 made from shape memory alloys having a transition temperature above body temperature, exhibit acceptable fatigue resistance. This is because there are no elastic forces exerted by the ligament 28, 128, 228 of the present invention, against the body. It is intended that a surgeon determine how much in-elastic resistance is necessary for each individual patient's needs and then pre-selects an assembly or device 10, 110, 210 at the time of surgery to ensure the best resistance. Preferably the ligament 28, 128, 228 is non-braided and is formed as a unitary contiguous member enabling the ligament to resist shear forces. In the instance where a surgeon may be supplementing fixation of two vertebral bodies 24, 26 and 124, 126 with a fusion cage (not shown), a flexible inelastic assembly or device 10, 110, 210 with pedicle screws 20, 22 and 120, 122 is preferable to limit motion and allow stress transfer through the fusion site in accordance to Wolfe's law. In this instance the surgeon would select a less flexible assembly or device 10, 110, 210 with a larger inelastic ligament 28, 128, 228 such as 5-6 mm in diameter. The diameter and length of the inelastic ligament 28, 128, 228 determine the flexibility of the surgical construct. In another instance a surgeon may selectively remove the facia from two adjacent vertebral bodies 24, 26 and 124, 126 to eliminate arthritis caused by bony contact at the facia. To replace the support for the vertebral bodies after the faciaectomy the surgeon would use a flexible inelastic assembly or device 10, 110, 210 with pedicle screws 20, 22 and 120, 122 to ensure that axial spacing between posterior segments (not shown) of vertebral bodies 24, 26 and 124, 126 is maintained. In this instance it would be preferable for the surgeon to select a more flexible assembly or device 10, 110, 210 that has an inelastic ligament 28, 128, 228 with a diameter closer to 3-4 mm. This surgical construct would allow a patient to have constrained motion but would limit contact between the facia of the two vertebral bodies 24, 26 and 124, 126. The rigid rod portions 42, 44 and 242, 244 are typically manufactured from stainless steel or titanium and are preferably in the diameter range of 4-7 mm. This size range is typical of other commercially available spinal implant hardware so that flexible inelastic assembly or device 10, 110, 210 of the present type is universally received by existing pedicle screws 20, 22 and 120, 122.

FIGS. 3-7 illustrate use of pedicle screws 20, 22 and 120, 122 to fasten the flexible assembly 10 or device 110 to vertebral bodies 24, 26 or 124, 126. However, other attachment means are possible as well as a variety of alternate locations for mounting. In a traditional spinal stabilization system the rods are placed posterior to and on either side of the spinous process. Depending on the pathology observed, a surgeon might select a unitary flexible ligament 128 or assembly 10 to mount posterior to the theoretical centerline of a patient between two spinous processes. Or alternatively a flexible inelastic ligament 128 or assembly 10 may be placed on the anterior side of the vertebral bodies 24, 26; 124, 126.

The present invention is by no means restricted to the above described preferred embodiments, but covers all variations that might be implemented by using equivalent functional elements or devices that would be apparent to a person skilled in the art, or modifications that fall within the spirit and scope of the appended claims.

What is claimed is:

1. An orthopedic device for stabilizing the spinal column between anchorage locations on respective first and second vertebral bodies, the device comprising:
an elongated bridge having first and second ends operatively connected at the respective anchorage locations, the bridge containing an implantable ligament selected to be inelastic at body temperature further capable of continuous plastic deformation to allow relative constrained motion while resisting shear forces exerted between the vertebral bodies.

2. The orthopedic device of claim 1 wherein the implantable ligament is in the form of a wire, tube or band.

3. The orthopedic device of claim 1 further comprising a compression dampening member.

4. The orthopedic device of claim 1 further comprising a tension dampening member.

5. The orthopedic device of claim 1 wherein the bridge member further comprises an assembly including at least two rigid rod members each correspondingly retained with an end of the ligament, and independently attached to the vertebral bodies at the respective anchorage locations.

6. The orthopedic device of claim 1 further comprising a plate segment retained with an end of the ligament and independently attached to a vertebral body at an anchorage location.

7. The orthopedic device of claim of claim 1 wherein the ligament further comprises a nickel titanium alloy selected to be in-elastic at body temperature.

8. An orthopedic device for stabilizing the spinal column between respective first and second vertebral bodies, the device comprising:
an elongated ligament with opposed first and second ends, the ligament partially formed of an implantable nickel titanium alloy capable of continuous plastic in-elastic deformation at body temperature;
a rigid rod member retained with the first end and secured to the first vertebral body, the second end operatively fixed with the second vertebral body, wherein plastic deformation in the ligament allows relative constrained motion between the vertebral bodies.

9. The orthopedic device of claim 8 further comprising a second rigid rod member retained with the second end and secured to the second vertebral body.

10 The orthopedic device of claim 9 further comprising first and second anchoring screws respectively attached to first and second rigid rod members.

11. The orthopedic device of claim 10 further comprising a compression dampening member sandwiched between the first and second rigid rod members.

12. The orthopedic device of claim 11 wherein the compression dampening member surrounds the ligament.

13. An orthopedic device for stabilizing the spinal column between anchorage locations on respective first and second vertebral bodies, the device comprising:
a plurality of screws;
an elongated ligament with opposed first and second ends respectively connected to the plurality of screws, the ligament partially formed of an implantable nickel titanium alloy capable of continuous plastic in-elastic deformation at body temperature; and
a compression dampening member sandwiched between the screws and surrounding the ligament, wherein
plastic deformation in the ligament allows relative constrained motion between the vertebral bodies.

14. An orthopedic device for stabilizing the spinal column comprising:
an elongated ligament with two enlarged end portions, the ligament partially formed of an implantable nickel titanium alloy capable of continuous plastic inelastic deformation at body temperature;
two rigid rod members each containing a bore sized for the ligament, the rigid rod members retained with either end of the ligament and engageable with two vertebral bodies by a plurality of anchors;
a compression dampening member surrounding the ligament and sandwiched between the rigid rods; and
two tension dampening members captured within the bores, surrounding the ligament and abutting the enlarged end portions respectively; wherein
plastic deformation in the ligament allows for relative constrained motion between the vertebral bodies.

15. A surgical kit comprising:
at least one bone anchor; and
a flexible spine stabilization device further comprised of a ligament partially formed of an implantable nickel titanium alloy capable of continuous plastic inelastic deformation at body temperature,

16. The surgical kit of claim 15 wherein the device further comprises at least one rigid fusion rod.
